(19) United States
(12) Patent Application Publication
(10) Pub. No.: US 2009/0118642 A1
(43) Pub. Date: May 7, 2009

(54) URINE STREAM COLLECTION UNIT

(76) Inventor: Charles M. Washington, Roscoe, IL (US)

Correspondence Address: LEYDIG VOIT & MAYER, LTD.;(ROCKFORD OFFICE), TWO PRUDENTIAL PLAZA, SUITE 4900, 180 NORTH STESTON AVENUE, CHICAGO, IL, 60601-6731, US

(21) Appl. No.: 12/258,954

(22) Filed: Oct. 27, 2008

Related U.S. Application Data
(60) Provisional application No. 60/985,486, filed on Nov. 5, 2007.
(60) Provisional application No. 61/070,607, filed on Mar. 24, 2008.

(51) Int. Cl. A61B 5/20

(52) U.S. Cl. 600/574

(57) ABSTRACT

A urine stream collection unit. The collection unit includes a receptacle cup, having a wall surrounding a fluid containment portion. An elongate handle structure extends away from the receptacle cup. The elongate handle structure includes a platform segment disposed at a distal end of the elongate handle structure. The platform segment is characterized by an enhanced width relative to an adjacent portion of the elongate handle structure.

### CROSS-REFERENCE TO RELATED APPLICATIONS

This non-provisional application claims the benefit of U.S. provisional application 60/985,486 filed Nov. 5, 2007 and U.S. provisional application 61/070,607 filed Mar. 24, 2008 the contents of all of which are hereby incorporated by reference in their entirety as if fully set forth herein.

### TECHNICAL FIELD

This patent disclosure relates generally to urine specimen collection units and, more particularly, to urine stream collection units adapted for mid-stream collection of specimens by persons having impaired access to the urethra opening due to obesity and/or other physical disability. A device consistent with the present disclosure may be used to provide both a cleaning function and a collection function.

### BACKGROUND

Urinalysis is a common medical testing procedure used to evaluate various bodily functions. In order to conduct proper analytical testing it is generally desirable to first clean the area surrounding the urethra opening and to collect a so called “mid-stream” urine sample at an intermediate stage of the urination event. Such cleaning and mid-stream sampling provides the benefit of reducing extraneous debris in the sample.

Urine samples are typically collected by the patient using a hand-held sample collection cup. Such a collection method can be carried out by most patients. However, for some percentage of female patients, cleaning of the area surrounding the urethra opening and then collecting the specimen is impractical due to an inability to reach between the legs to clean the area and then hold the collection cup during the urination event. Such difficulty may be particularly acute in patents who suffer from obesity or who have limited use of their arms due to arthritis or other disabling conditions.

### SUMMARY

The present invention offers advantages and alternatives over the prior practices by providing a urine stream collection unit having a receptacle and an operatively connected elongate handle structure which may be held at a remote position in front of the user. The receptacle may be placed in position adjacent the urethra opening by the patient manipulating the handle structure. Thus, the patient is not required to reach between the legs. The handle structure may include a platform segment at the terminal end to support a removable wiping element such as an adhesive-backed feminine hygiene pad or the like for use in cleaning the area surrounding the urethra opening prior to taking the sample.

### BRIEF DESCRIPTION OF THE DRAWINGS

The following drawings which are incorporated in and which constitute a part of this specification illustrate several exemplary embodiments and constructions consistent with this disclosure wherein:

FIG. 1 is a perspective view of an exemplary urine stream collection unit adapted for remote manipulation;

FIG. 2 is a side view of the exemplary collection unit of FIG. 1;

FIG. 3 is a view illustrating an exemplary handle platform segment with a removably attached wiper;

FIG. 4 illustrates a user manipulating the exemplary collection unit of FIG. 1 into position to clean the urethra area prior to sampling; and

FIG. 5 illustrates a user manipulating the exemplary collection unit of FIG. 1 into position to collect a urine sample.

### DETAILED DESCRIPTION

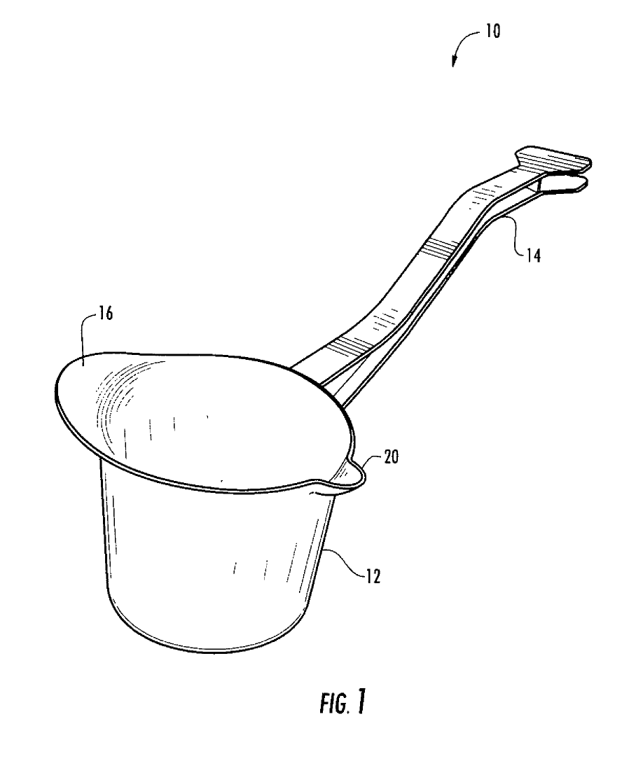

Reference will now be made to the drawings, wherein to the extent possible, like elements are designated by like reference numerals throughout the various views. FIG. 1 illustrates an exemplary urine stream collection unit 10. As shown, the collection unit 10 includes a receptacle cup 12 operatively connected to a handle 14. According to one contemplated practice, the receptacle cup 12 may be integrally formed with the handle 14 from a material such as plastic or the like. However, it is likewise contemplated that the components may be formed separately and thereafter joined if desired. It is also contemplated that materials other than plastic may be used if desired.

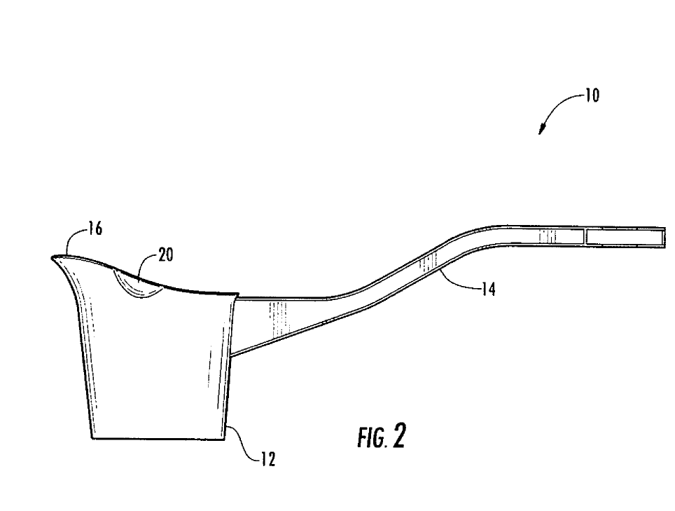

As best seen through joint reference to FIGS. 1 and 2, the receptacle cup 12 includes a wall surrounding a fluid containment cavity. The receptacle cup 12 includes a raised angled lip portion 16 which extends partially around the circumference of the receptacle cup 12 and which projects generally away from the handle 14. The raised angled lip portion 16 provides an enhanced collection target zone for the urine stream while reducing splashing. The receptacle cup 12 may also include a pour spout 20 projecting away from one side in generally transverse orientation to the handle 14. As will be appreciated, the pour spout 20 may be used to transfer the specimen to another container for subsequent processing.

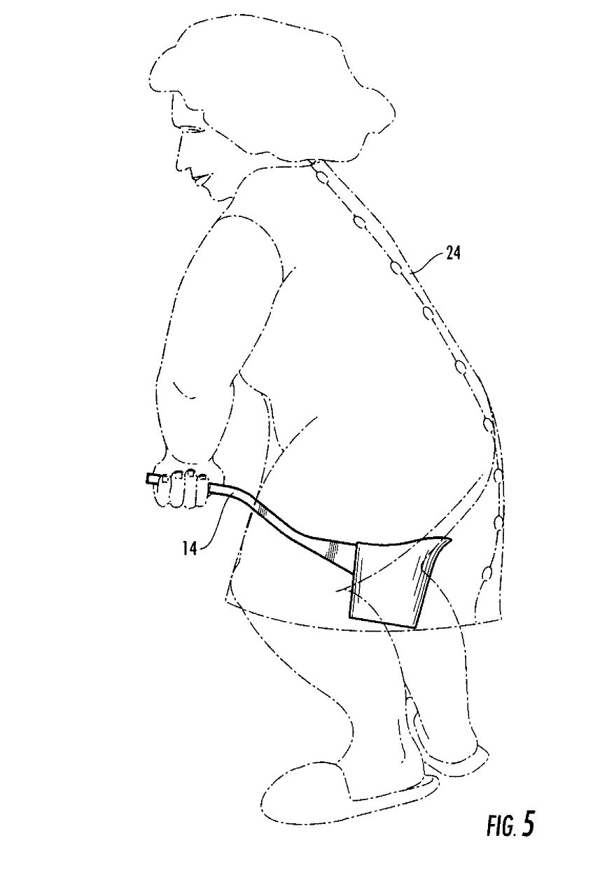

As illustrated, in FIG. 2, the handle 14 is angled generally upwardly such that the grip portion is elevated relative to the receptacle cup 12. This enhanced elevation assists the user 24 in manipulating the receptacle cup 12 into position between the legs while holding the handle 14 at an elevated forward position (FIG. 5).

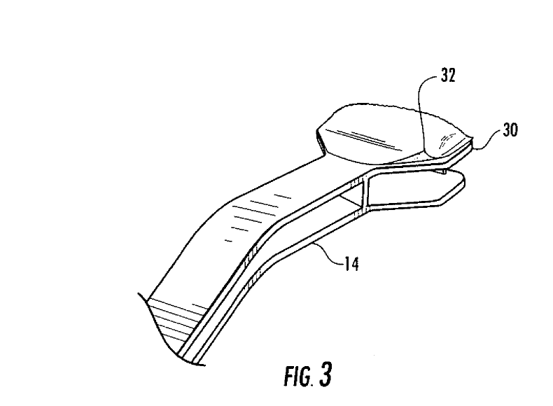

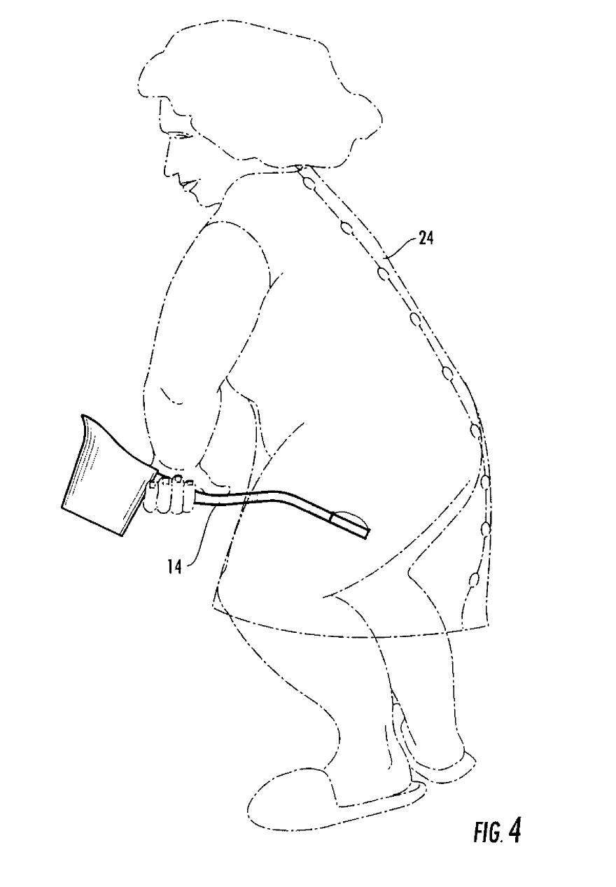

As best seen through joint reference to FIGS. 3 and 4, the handle 14 may include an enhanced width platform segment 30 at the terminal end remote from the receptacle cup 12. This enhanced width platform segment 30 may provide a base for placement of a wiping element 32 such as an adhesive-backed feminine hygiene pad or the like. In practice, the wiping element 32 may be secured in place across the enhanced width platform segment 30. Prior to the collection of the sample, the urine stream collection unit 10 may be oriented with the platform segment 30 located between the legs of the user 24 such that the wiping element 32 may be used to clean the area surrounding the urethra opening. The wiping element 32 may then be removed and thrown away. Such cleaning prior to sampling aids in providing a clean, debris-free specimen for subsequent analysis.

Of course, it is contemplated that any number of other structures and combinations may likewise be utilized. In this regard, it will be appreciated that the foregoing description and drawings provide exemplary embodiments. However, it is contemplated that other implementations of the disclosure may differ in detail from the foregoing examples. Accordingly, this disclosure contemplates the inclusion of all modifications and equivalents of the subject matter disclosed herein as permitted by applicable law. Moreover, any combination of the above-described elements in all possible variations thereof is contemplated unless otherwise indicated herein or otherwise clearly contradicted by context.

What is claimed is:

1. A urine stream collection unit comprising:
a receptacle cup, said receptacle cup including a wall surrounding a fluid containment portion, the wall including a raised angle lip portion extending at least partially around the fluid containment portion, the raised angle lip portion projecting upwardly and away from the fluid containment portion, a pour spout projecting away from an edge portion of the wall in transverse orientation to the raised angle lip portion; and
an elongate handle structure extending away from said receptacle cup in substantially opposing relation to the raised angle lip portion, the elongate handle structure including a platform segment disposed at a distal end of the elongate handle structure, the platform segment being characterized by an enhanced width relative to an adjacent portion of the elongate handle structure.

2. The urine stream collection unit as recited in claim 1, wherein the receptacle cup is integral with the elongate handle structure.

3. The urine stream collection unit as recited in claim 1, wherein the receptacle cup and the elongate handle are plastic.

4. The urine stream collection unit as recited in claim 1, further including a disposable wiping element removably attached across the platform segment.

5. The method as recited in claim 4, wherein the wiping element is a feminine hygiene pad.

6. A urine stream collection unit comprising:
a receptacle cup, said receptacle cup including a wall surrounding a fluid containment portion, the wall including a raised angle lip portion extending at least partially around the fluid containment portion, the raised angle lip portion projecting upwardly and away from the fluid containment portion, a pour spout projecting away from an edge portion of the wall in transverse orientation to the raised angle lip portion; and
an elongate handle structure extending away from said receptacle cup in substantially opposing relation to the raised angle lip portion, the elongate handle structure including an platform segment disposed at a distal end of the elongate handle structure, the platform segment being characterized by an enhanced width relative to an adjacent portion of the elongate handle structure, wherein the elongate handle structure includes a first segment in connected relation to the receptacle cup, a second segment extending in angled relation upwardly from the first segment at a first angle and a third segment extending away from the second segment towards the enhanced width platform segment at a reduced angle relative to said first angle.

7. The urine stream collection unit as recited in claim 6, wherein the receptacle cup is integral with the elongate handle structure.

8. The urine stream collection unit as recited in claim 6, wherein the receptacle cup and the elongate handle are plastic.

9. The urine stream collection unit as recited in claim 6, further including a disposable wiping element removably attached across the platform segment.

10. The method as recited in claim 9, wherein the wiping element is a feminine hygiene pad.

11. A method of collecting a urine sample, using a urine stream collection unit including a receptacle cup, said receptacle cup including a wall surrounding a fluid containment portion, the wall including a raised angle lip portion extending at least partially around the fluid containment portion, and an elongate handle structure extending away from said receptacle cup in substantially opposing relation to the raised angle lip portion, the elongate handle structure including a platform segment disposed at a distal end of the elongate handle structure, the platform segment being characterized by an enhanced width relative to an adjacent portion of the elongate handle structure, the method comprising:
applying a removable wiping element across the platform segment;
cleaning an area surrounding a urethra opening using the wiping element in attached relation to the platform segment;
positioning the receptacle cup in substantially opposing relation to the urethra opening; and
collecting a portion of a urine stream discharged from the urethra opening.

12. The method as recited in claim 11, wherein the wiping element is a feminine hygiene pad.
